SEQUENCE LISTING (17 sequences)

SEQ ID NO: 1 - RNA, 20 nt, synthetic construct
  misc_feature 1..20: note = Synthetic Polynucleotide
  source 1..20: mol_type = other RNA
ggggttcggctgccgggaag

SEQ ID NO: 2 - RNA, 20 nt, synthetic construct
  misc_feature 1..20: note = Synthetic Polynucleotide
  source 1..20: mol_type = other RNA
ggaagaggcgcgggtagaag

SEQ ID NO: 3 - RNA, 20 nt, synthetic construct
  misc_feature 1..20: note = Synthetic Polynucleotide
  source 1..20: mol_type = other RNA
gtagcaagctctggaactca

SEQ ID NO: 4 - RNA, 20 nt, synthetic construct
  misc_feature 1..20: note = Synthetic Polynucleotide
  source 1..20: mol_type = other RNA
tgctctcacagtactcgctg

SEQ ID NO: 5 - RNA, 20 nt, synthetic construct
  misc_feature 1..20: note = Synthetic Polynucleotide
  source 1..20: mol_type = other RNA
tcgaaatgcagagagtggtg

SEQ ID NO: 6 - RNA, 20 nt, synthetic construct
  misc_feature 1..20: note = Synthetic Polynucleotide
  source 1..20: mol_type = other RNA
aatggggatcgcagcacata

SEQ ID NO: 7 - DNA, 650 bp, synthetic construct
  misc_feature 1..650: note = Synthetic
  source 1..650: mol_type = other DNA
tgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctc

SEQ ID NO: 8 - DNA, 650 bp, synthetic construct
  misc_feature 1..650: note = Synthetic
  source 1..650: mol_type = other DNA
gaggcgaaagcccgacacccagcttcggtcagagaaatgagagggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgccccggccccggccccggcccctagcgcgcgactcctgagttccagagcttgctacaggctgcggttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcctagcgggacaccgtaggttacgtctgtctgttttctatgtgcgatgacgttttctcacgaggctagcgaaatggggcggggca

SEQ ID NO: 9 - DNA, 287 bp, synthetic construct
  misc_feature 1..287: note = Synthetic
  source 1..287: mol_type = other DNA
aagaaaacaaggagggaaacaaccgcagcctgtagcaagctctggaactcaggagtcgcgcgctaggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccc

SEQ ID NO: 10 - DNA, 287 bp, synthetic construct
  misc_feature 1..287: note = Synthetic
  source 1..287: mol_type = other DNA
gggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgccccggccccggcccctagcgcgcgactcctgagttccagagcttgctacaggctgcggttgtttccctccttgttttctt

SEQ ID NO: 11 - DNA, 230 bp, synthetic construct
  misc_feature 1..230: note = Synthetic
  source 1..230: mol_type = other DNA
aagaaaacaaggagggaaacaaccgcagcctgtagcaagctctggaactcaggagtcgcgcgctaggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccggga

SEQ ID NO: 12 - DNA, 35 bp, synthetic construct
  misc_feature 1..35: note = Synthetic
  source 1..35: mol_type = other DNA
aagaaaacaaggagggaaacaaccgcagcctgtaa

SEQ ID NO: 13 - DNA, 34 bp, synthetic construct
  misc_feature 1..34: note = Synthetic
  source 1..34: mol_type = other DNA
aagaaaacaaggagggaaacaaccgcagcctgta

SEQ ID NO: 14 - DNA, 23 bp, synthetic construct
  misc_feature 1..23: note = Synthetic
  source 1..23: mol_type = other DNA
gaacaggtacttctcagtgatgg

SEQ ID NO: 15 - DNA, 360 bp, synthetic construct
  misc_feature 1..360: note = Synthetic
  source 1..360: mol_type = other DNA
ggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagct

SEQ ID NO: 16 - DNA, 360 bp, synthetic construct
  misc_feature 1..360: note = Synthetic
  source 1..360: mol_type = other DNA
agctttcaacagataggttaacatgattaataagctgaaaaatcacttaccttatgcatccatattcttcctttccggattatatgtgttaatctatcaacacacactctatgaagtgggaggtagaaactaagttctgtctgtggaagtataattgatagtccatatgtgctgcgatccccattccagtttccatcaaagattaatgaaacaataatcactcccttttcagacaagacaaaaaactttacatctatagcaccactctctgcatttcgaaggatttctccatttagagtgtggttggcaagaaaagttatttctccatcactgagaagtacctgttctgtctttggagcc

SEQ ID NO: 17 - DNA, 26 bp, synthetic construct
  misc_feature 1..26: note = Synthetic
  source 1..26: mol_type = other DNA
ccttatgcatccatattcttcctttc

(19) United States
(12) Patent Application Publication
(10) Pub. No.: US 2024/0167025 A1
(43) Pub. Date: May 23, 2024

(54) METHODS OF TREATING AMYOTROPHIC LATERAL SCLEROSIS (ALS)

(71) Applicant: University of Massachusetts, Boston, MA (US)

(72) Inventors: Christian Mueller, Worcester, MA (US); Abbas Abdallah, Worcester, MA (US)

(73) Assignee: University of Massachusetts, Boston, MA (US)

(21) Appl. No.: 18/505,163

(22) Filed: Nov. 9, 2023

Related U.S. Application Data
(63) Continuation of application No. 16/611,581, filed on Nov. 7, 2019, now Pat. No. 11,859,179.
(60) Provisional application No. 62/503,909, filed on May 9, 2017.

(51) Int. Cl. C12N 15/11; A61K 35/76; C12N 9/22; C12N 15/86; C12N 15/861; C12N 15/90

(52) U.S. Cl. CPC C12N 15/11; A61K 35/76; C12N 9/22; C12N 15/86; C12N 15/8616; C12N 15/907; C12N 2310/20; C12N 2750/14143; C12N 2750/14171; C12N 2800/80

(57) ABSTRACT

Aspects of the disclosure relate to recombinant gene editing complexes comprising a recombinant gene editing protein and guide RNA (gRNA) that specifically hybridizes to a region of a C90RF72 gene (e.g., a region flanking a G₄C₂ repeat or within a exonic region of the gene).

### RELATED APPLICATIONS

This Application claims the benefit under 35 U.S.C. 119(e) of the filing date of U.S. Provisional Application Ser. No. 62/503,909 filed on May 9, 2017, the entire contents of which are incorporated herein by reference.

### BACKGROUND

Amyotrophic Lateral Sclerosis (ALS) is a fatal neurodegenerative disease that is characterized by progressive loss of motor neurons, both in the brain (upper motor neurons) and the spinal cord (lower motor neurons). The average age of onset is in the late 50s-60s and the patients succumb to death in 3-5 years. The current estimated prevalence in the United States is 1 in 50,000 people. ALS is grouped into two categories depending on whether the disease is inherited or not; about 50-10% of cases are familial ALS and the remaining percentage falls under sporadic ALS. Mutations in more than 25 genes have been linked to ALS since the discovery of SOD1.

### SUMMARY

Aspects of the disclosure relate to methods and compositions for treating ALS. Some aspects relate to a (GGGGCC)ₙ repeat expansion in the non-coding region of the C9orf72 gene, which is a major cause for both familial (25-40%) and sporadic (7%) ALS. In some embodiments, the repeat expansion may lead to haploinsufficiency due to reduced C9orf72 transcript levels and/or reduced activity or function of C9orf72 gene products. In some embodiments, the repeat expansion may lead to nuclear RNA foci formation which leads to RNA and RNA binding protein sequestration. In some embodiments, the repeat expansion may lead to toxic dipeptide proteins produced through repeat-associated non ATG (RAN) translation. The disclosure is based, in part, on gene editing molecules (e.g., RNAs, such as guide RNAs (gRNAs), trans-activating crRNA (tracrRNA), etc., proteins, such as CRISPR/Cas proteins, etc., and complexes of RNAs and CRISPR/Cas proteins) that direct cleavage, excision, or degradation of (GGGCC)ₙ repeat expansions in a C9orf72 gene. Accordingly, some aspects of the disclosure relate to methods for treating C9FTD/ALS that involve editing (e.g., physically erasing) the repeat expansions from the C9ORF72 genomic locus to restore the gene to a normal or healthy state.

In some embodiments, methods provided herein involve use of CRISPR/Cas9-guided genome editing or related systems. In some embodiments, CRISPR/Cas9 functions as a nuclease that can make double-strand breaks in genomic DNA. In some embodiments, CRISPR/Cas9 is guided to a target sequence by an associated guide RNA, e.g., with ˜20 nucleotides of complementarity to the target sequence. In some embodiments, CRISPR/Cas9 related methods provided herein involve delivery of the Cas9 enzyme with a guide RNA via one or more AAV vectors.

In some embodiments, methods provided herein alleviate the cause of ALS in patients with C9orf72 specific mutations. Further aspects of the disclosure relate to methods for targeting (e.g., using gene editing systems (e.g., CRISPR/Cas9)) the repeat expansion in the intronic region without affecting any of the exons. In some embodiments, guide RNAs have been developed that are capable of directing the removal of the repeat region using CRISPR Cas9 system. In some embodiments, the RNA guides are packaged into rAAV vectors (e.g., rAAV9 vectors) for in vivo delivery. In some embodiments, gene editing occurs in primary neurons in culture. In some embodiments, gene editing occurs in animals in vivo, e.g., in mice through tail vein injections.

Accordingly, in some aspects, the disclosure provides an isolated nucleic acid comprising the sequence set forth in any one of SEQ ID NOs: 1 to 6, or a sequence complementary in any one of them.

In some aspects, the disclosure provides an isolated nucleic acid comprising a nucleic acid sequence encoding a guide RNA (gRNA) having the sequence set forth in any one of SEQ ID NOs: 1-6, or a sequence complementary to any one of them.

In some embodiments, an isolated nucleic acid sequence is flanked by adeno-associated virus (AAV) inverted terminal repeats (ITRs). In some embodiments, AAV ITRs are AAV2 ITRs, AAV3 ITRs, AAV4 ITRs, AAV5 ITRs, AAV6 ITRs, AAV7 ITRs, AAV8 ITRs, or AAV9 ITRs.

In some aspects, the disclosure provides an isolated nucleic acid comprising a transgene encoding two or more guide RNAs (gRNAs) that specifically hybridize to a target nucleic acid sequence flanking opposite sides of a G₄C₂ repeat of a C9ORF72 gene, flanked by adeno-associated virus (AAV) inverted terminal repeats (ITRs).

In some embodiments, two or more gRNAs each comprise or consist of the sequence set forth in any one of SEQ ID NOs: 1-4, or a sequence complementary to any one of them.

In some embodiments, a transgene encodes a first gRNA having the sequence set forth in SEQ ID NO: 1 and a second gRNA having the sequence set forth in SEQ ID NO: 3. In some embodiments, a transgene encodes a first gRNA having the sequence set forth in SEQ ID NO: 2 and a second gRNA having the sequence set forth in SEQ ID NO: 3.

In some embodiments, a transgene comprises a promoter. In some embodiments, a promoter is a CB promoter.

In some aspects, the disclosure provides a recombinant adeno-associated virus (rAAV) comprising an isolated nucleic acid as described by the disclosure; and at least one AAV capsid protein.

In some embodiments, a capsid protein is of a serotype selected from AAV1, AAV2, AAV3, AAV4, AAV5, AAV6, AAV7, AAV8, AAV9, or a variant of any of the foregoing. In some embodiments, a capsid protein is an AAV9 capsid protein.

In some aspects, the disclosure provides a composition comprising an rAAV as described by the disclosure, and a recombinant gene editing protein. In some embodiments, a recombinant gene editing protein is encoded by an rAAV vector. In some embodiments, a recombinant gene editing protein is a CRISPR/Cas protein, optionally a Cas9 protein.

In some aspects, the disclosure provides a mammalian cell expressing: two or more guide RNAs (gRNAs) that specifically hybridize to a target nucleic acid sequence flanking opposite sides of a G₄C₂ repeat of a C9ORF72 gene; and a recombinant gene editing protein that interacts with the two or more gRNAs.

In some embodiments, a recombinant gene editing protein is a CRISPR/Cas protein. In some embodiments, a recombinant gene editing protein is a Cas protein selected from Cas9, Cas6, and Cpf1. In some embodiments, a recombinant gene editing protein is Cas9.

In some embodiments, each of the gRNAs comprises the sequence set forth in any one of SEQ ID NOs: 1 to 4, or a sequence complementary to any one of them.

In some embodiments, a mammalian cell expresses 2, 3, or 4 gRNAs that each specifically hybridizes to a target nucleic acid sequence flanking opposite sides of a G₄C₂ repeat of a C9ORF72 gene.

In some embodiments, a mammalian cell expresses a first gRNA having the sequence set forth in SEQ ID NO: 1 and a second gRNA having the sequence set forth in SEQ ID NO: 3.

In some embodiments, a mammalian cell expresses a first gRNA having the sequence set forth is SEQ ID NO: 2 and a second gRNA having the sequence set forth in SEQ ID NO: 3.

In some embodiments, a mammalian cell further expresses a trans-activating crRNA (tracrRNA).

In some embodiments, a target nucleic acid sequence is positioned in a non-protein-coding region between Exon 1b and Exon 2 of the C9ORF72 gene, or is positioned in a non-protein-coding region between Exon 2 and Exon 3 of the C9ORF72 gene.

In some aspects, the disclosure provides a method comprising delivering to a cell: a recombinant gene editing protein: and two or more guide RNAs (gRNAs) that specifically hybridize to target nucleic acid sequences flanking opposite sides of a G₄C₂ repeat of a C9ORF72 gene.

In some embodiments, delivery of a recombinant gene editing protein and gRNAs to a cell results in removal of the G₄C₂ repeat from at least one allele of the C9ORF72 gene in the cell.

In some embodiments, a recombinant gene editing protein and/or gRNAs are delivered to a cell using a recombinant AAV vector comprising a nucleic acid engineered to express the protein or gRNAs in the cell.

In some embodiments, a cell is in vivo. In some embodiments, a cell is a primary neuron.

In some embodiments, a recombinant AAV vector comprises an AAV9 capsid protein or variant thereof.

In some embodiments, a gRNA comprises a sequence selected from SEQ ID NO: 1-4 or a sequence complementary to any one of them.

In some embodiments, the disclosure provides a mammalian cell expressing a guide RNA (gRNA) that specifically hybridizes to an exonic region of a C9ORF72 gene; and a recombinant gene editing protein that interacts with the gRNA.

In some embodiments, a recombinant gene editing protein is a CRISPR/Cas protein. In some embodiments, a recombinant gene editing protein is a Cas protein selected from Cas9, Cas6, and Cpf1. In some embodiments, a recombinant gene editing protein is Cas9.

In some embodiments, a gRNA comprises the sequence set forth in SEQ ID NO: 5 or 6, or a sequence complementary to either one of them.

In some embodiments, a mammalian cell further comprises a trans-activating crRNA (tracrRNA).

In some embodiments, interaction of a gRNA and a recombinant gene editing protein results in formation of a complex, and binding of the complex to the C9ORF72 gene results in non-sense mediated decay of the C9ORF72 gene.

In some aspects, the disclosure provides a method of reducing RNA foci and/or dipeptide formation in a cell, the method comprising expressing in the cell a recombinant gene editing complex comprising a guide RNA (gRNA) that specifically hybridizes to an exonic region of a C9ORF72 gene and a recombinant gene editing protein that interacts with the gRNA, wherein delivery of the recombinant gene editing complex to the cell results in insertions or deletions in the C9ORF72 gene that lead to non-sense mediated decay of C9orf72 transcripts transcribed from the gene.

In some embodiments, a recombinant gene editing protein and/or gRNA(s) of a complex are expressed in a cell using a recombinant AAV vector comprising a nucleic acid engineered to express the protein or gRNAs in the cell.

In some embodiments, a cell is in vivo. In some embodiments, a cell is a primary neuron.

In some embodiments, a recombinant AAV vector comprises as AAV9 capsid protein or variant thereof.

In some embodiments, a gRNA comprises a sequence selected from SEQ ID NO: 5 or 6, or a sequence complementary to either one of them.

In some embodiments, the disclosure provides a method comprising delivering to a cell: a guide RNA (gRNA) that specifically hybridizes to one or more exonic regions of a C9ORF72 gene; and a recombinant gene editing protein that interacts with the gRNA.

In some embodiments, the method further comprises delivering to the cell two guide RNAs that specifically hybridize to different positions within the same exon of a C9ORF72 gene.

In some embodiments, an exonic region is within exon 3 of the C9ORF72 gene.

In some embodiments, a recombinant gene editing protein and/or gRNA(s) is/are delivered to a cell using a recombinant AAV vector comprising a nucleic acid engineered to express the protein or gRNAs in the cell.

In some embodiments, delivery of a recombinant gene editing protein and gRNAs to a cell results in insertions or deletions in the C9ORF72 gene that lead to non-sense mediated decay of C9orf72 transcripts transcribed from the gene.

In some embodiments, a gRNA comprises a sequence selected from SEQ ID NO: 5 or 6, or a sequence complementary to either one of them.

In some embodiments, a recombinant gene editing protein is a Crisper/Cas9 protein.

In some aspects, the disclosure provides a recombinant gene editing complex configured to remove all or a portion of the G₄C₂ repeat from at least one allele of a C9ORF72 gene in a cell or to induce an insertion or deletion within an exonic region of the C9ORF72 gene in the cell that results in non-sense mediated decay of C9orf72 transcripts transcribed from the gene.

In some embodiments, the disclosure provides a method comprising delivering to a cell: one or more guide RNAs (gRNAs) that specifically hybridize to target nucleic acid sequences flanking opposite sides of a G4C2 repeat of a C9ORF72 gene; or one or more guide RNAs (gRNAs) that specifically hybridize to one or more exonic regions of a C9ORF72 gene.

In some embodiments, a cell expresses a recombinant gene editing protein that binds to one or more guide RNAs (gRNAs).

### BRIEF DESCRIPTION OF DRAWINGS

FIGS. 1A to 1C show guide RNAs described in Example 1. FIG. 1A shows the human C9orf72 (NG_031977.1) gene sequence surrounding the G₄C₂ expansion repeat. RNAs r9 (also referred to as “gr-r9” or “gRNA 2”), f11 (also referred to as “gr-f11” or “gRNA 3”), and r1=(also referred to as “gr-r1” or “gRNA 4”); PCR primers C9Var1-f and C9In1-R. FIG. 1B shows a schematic representation of the C9 region containing the (GGGCC)ₙ expansion. Relative positioning of the repeat expansion of G₄C₂, non-coding Exon 2, RNA guides gr-r9, gr-r1, and gr-f11, editing primers C9Var1-f and C9Ind1-R, no editing forward primer NoE-F1, and the repeat primed PCR primer RP-PCR-R is shown. FIG. 1C shows design and testing of gRNAs in HEK 293 cells. Since the repeat expansion is close to exon 2, the only efficient guide on the 3′ end would also span exon 2 (which is un translated). In HEK 293 cells, there is only 3 “GGGGCC” repeats-successful editing will reduce the size of the PCR product using the two indicated primers from 520 bp to around 315 bp. Vectors containing gRNA combination 2-3 and 2-4 were the most efficient and were subsequently packaged in AAV9 capsid protein.

FIGS. 2A to 2B show data on Cas9-mediated C9orf72 G₄C₂ editing. FIG. 2A shows agarose electrophoresis of PCR products amplified by C9Var1-f and C9In1-R primers. Unedited PCR product size is 523 bp; edited PCR product size for gRNA 1 & 4 (f1+r1) and gRNA 1 & 3 (f1+f11) is ˜250 bp; edited PCR product by gRNA 2 & 3 (r9+f11) and gRNA 2 & 4 (r9+r1) is ˜320 bp (+/− several base pairs with indels). FIG. 2B shows an alignment of the sequence of the PCR products gel extracted from FIG. 2A (indicated with arrows).

FIG. 3 shows Cas9-mediated C9orf72 G₄C₂ editing in mouse primary neurons. Agarose electrophoresis of PCR products amplified by C9Var1-f and C9In1-R primers. The edited PCR products appear around 320 bp, while the unedited DNA is not amplified.

FIG. 4 shows Cas9-mediated C9orf72 G₄C₂ editing in vivo confirmed through regular PCR. Agarose electrophoresis of PCR products amplified by C9Var1-f and C9In1-R primers or NoE-F1 and C9In1-R combined in the same well. The edited PCR products are at ˜320 bp while unedited PCR products are at ˜120 bp.

FIGS. 5A to 5D show Cas9-mediated C9orf72 G₄C₂ editing in vivo confirmed through Repeat Primed PCR. Electropherograms of Repeat primed PCR products were run through a fragment analyzer and plotted using peak scanner software. The PCR reactions were run using DNA from BAC436 mice tail vein injected with either AAV9 SOD1 guide RNA (FIG. 5A), AAV9-CB-GFP-C9gR flank r9-r1 (FIG. 5B), AAV9-CB-GFP-C9gR flank r9-f11 (FIG. 5C) or uninjected wild type C57BL mice that don't express human C9 (FIG. 5D).

FIGS. 6A to 6C show representative data described in Example 2. FIG. 6A shows human C9orf72 gene sequence of exon 3. The locations of non-sense mediated decay (NMD) guide RNA 1r and 2f and the location and sequence of PCR indel analysis primers C9NMD Indel F1 and R1 are indicated. FIG. 6B shows agarose gel electrophoresis of PCR products amplified by C9NMD-Indel F1 and R1 PCR primers. HEK293T cells were transfected with LV-SpCas9 (Control) or LV-NMDgR-SpCas9 plasmid (2 μg) in triplicate. FIG. 6C shows digital droplet PCR (ddPCR) analysis of C9orf72 RNA level in cells from FIG. 6B. All variants of C9orf72 are detected with this particular probe-primer set. (Input RNA−10 ng per sample)* p<0.001.

FIG. 7 shows representative data for gene editing in mice injected via tail vein. Guide strands were tested through tail vein injection of BAC111 mice expressing both C9/Cas9 to determine whether they are functional in vivo. The liver of injected mice were dissected and genome DNA was extracted and a two PCR reactions were run. The top panel indicates gene editing occurs after injection of gRNA2-4 and gRNA2-3 but not PBS or SOD1-gRNA. As depicted in the bottom panel, one reaction (using primers C9Var1F and C9IndR) amplifies only edited DNA, since the repeat is GC rich and a polymerase cannot amplify through the repeat; thus, a band indicates edited DNA. The other reaction (using primers NoE-F1 and C9IndR) can only amplify unedited DNA.

FIGS. 8A to 8B show gene editing in cultured primary neurons from BAC111 expressing C9orf72 and Cas9. FIG. 8A shows fluorescence micrographs of neurons infected with PBS. AAV9-scGFP, AAV9-ROSA-tRFP, AAV9-gRNA 2 & 3, or AAV9-gRNA 2-4. FIG. 8B shows PCR amplification of edited DNA from cultured neurons amplified with C9Var1-F & NoER2 primers, as well as amplification of non-edited DNA using primers NoE-F1 and NoER2. Intensity of the hand amplified by the second set of primers was significantly less in the edited samples.

FIG. 9 shows direct visualization and quantification of gRNAs bound to unedited DNA from primary cultured neurons isolated from BAC111 mice expressing C9/Cas9 by fluorescence in-situ hybridization (FISH). Almost 55-60% of unedited cells have foci many with more than 10 foci. Edited cells exhibit foci in about 35-40% of cells, and the number of foci is dramatically reduced as well.

FIGS. 10A to 10B show gene editing in cultured primary neurons from BAC111 expressing C9orf72, but not Cas9. FIG. 10A shows fluorescence of neurons infected with Cas9, AAV9-ssGFP+Cas9, AAV9-ROSA-tRFP+Cas9. AAV9-gRNA 2-3+Cas9, or AAV9-gRNA 2-4+Cas9. FIG. 10B shows PCR amplification of edited DNA from cultured neurons amplified with C9Var1-F & NoER2. Amplification bands occur only in edited cells (e.g., cells treated with AAV9 gRNA 2-3+Cas9, or AAV9-gRNA 2-4+Cas9).

FIG. 11 shows direct visualization and quantification of gRNAs bound to unedited DNA from primary cultured neurons isolated front BAC111 mice expressing C9 by FISH. Around 55-60% of cells have foci when unedited (Ca9 only, single stranded GFP, ROSA); edited cells are reduced to 35-40%. Both gRNA pairs result in a significantly different reduction.

FIG. 12 shows gene editing in vivo in BAC111 mice expressing C9/Cas9 injected with PBS, SOD gRNA (control). R9-r1 (gRNA 2 & 4), or R9-f11 (gRNA 2 & 3). Brain, muscle, and liver tissue samples taken after 8 weeks each demonstrated gene editing with gRNA 2 & 3 and gRNA 2 & 4 guides, but not PBS and control SOD gRNA.

FIG. 13 shows FISH data (sense direction) on frontal sections of CAC111 mice that were facially injected at p1-2. The top panel shows a fluorescence micrograph indicating a reduction in number of foci in edited cells compared to untreated and control cells. The bottom panel shows data indicating the reduction is consistent for heterozygous and homozygous mice.

FIGS. 14A-14B show gene editing through stereotaxic striatal brain injections in Baloh and BAC111 mice. FIG. 14A shows the injection site and the brain slice used for tissue isolation. FIG. 14B shows that injection of PBS+Cas9, ROSA-tRFP+Cas9, gRNA 2 & 3+Cas9, gRNA 2 & 4+Cas9 promotes gene editing in Baloh C9 mice and BAC111 C9/Cas9 mice.

### DETAILED DESCRIPTION

Through genetic linkage analysis of familial ALS patients, several genes have been identified to be risk factors for ALS. In the first intron of chromosome 9 open reading frame 72 (C9orf72), a large repeat expansion consisting of GGGGCC hexanucleotide has been identified in families of familial ALS patients. These microsatellite expansions can be transcribed in a bidirectional manner, producing both sense and antisense transcripts. The RNA transcripts accumulate in the nucleus of affected regions in the brain as RNA foci; moreover, repeat-associated non-ATG (RAN) translation of the transcripts leads to generation of dipeptide aggregates in the neuronal cytoplasm within the affected region. There is evidence indicating dipeptides and RNA foci may be toxic and may disrupt nucleocytoplasmic transport, autophagy, and immune response.

Provided herein are methods and related compositions useful for reducing or removing (e.g., completely removing) GGGGCC (e.g., G₄C₂) repeat expansions. In some embodiments, methods provided herein reduce the accumulation of RNA foci and dipeptide aggregates in the nucleus and cytoplasm, respectively. To accomplish this, a gene editing approach involving CRISPR/Cas9 nuclease and guide RNAs targeted at different regions of C9orf72 gene were used in some embodiments. In some embodiments, strategies are outlined to excise the GGGGCC repeat in both in vitro and in vivo mice models.

### Gene Editing Molecules

In some aspects, the disclosure provides a recombinant gene editing complex comprising: a recombinant gene editing protein; and, a nucleic acid encoding a guide RNA (gRNA) that specifically hybridizes to a target nucleic acid sequence within the C9ORF72 locus that are useful for excising all or a portion of a GGGGCC repeat expansion.

As used herein, “gene editing complex” refers to a biologically active molecule (e.g., a protein, one or more proteins, a nucleic acid, one or more nucleic acids, or any combination of the foregoing) configured for adding, disrupting or changing genomic sequences (e.g., a gene sequence), for example by causing one or more double stranded breaks (DSBs) in a target DNA. Examples of gene editing complexes include but are not limited to Transcription Activator-like Effector Nucleases (TALENs), Zinc Finger Nucleases (ZFNs), engineered meganuclease re-engineered homing endonucleases, the CRISPR/Cas system, and meganucleases (e.g., Meganuclease I-SceI). In some embodiments, a gene editing complex comprises proteins or molecules (e.g., recombinant gene editing proteins) related to the CRISPR/Cas system, including but not limited to Cas9, Cas6, Cpf1, CRISPR RNA (crRNA), trans-activating crRNA (tracrRNA), and variants thereof.

In some embodiments, a recombinant gene editing protein is a nuclease. As used herein, the terms “endonuclease” and “nuclease” refer to an enzyme that cleaves a phosphodiester bond or bonds within a polynucleotide chain. Nucleases may be naturally occurring or genetically engineered. Genetically engineered nucleases are particularly useful for genome editing and are generally classified into four families: zinc finger nucleases (ZFNs), transcription activator-like effector nucleases (TALENs), meganucleases (e.g., engineered meganucleases) and CRISPR-associated proteins (Cas nucleases). In some embodiments, the nuclease is a ZFN. In some embodiments, the ZFN comprises a FokI cleavage domain. In some embodiments, the ZFN comprises Cys₂His₂ fold group. In some embodiments, the nuclease is a TALEN. In some embodiments, the TALEN comprises a FokI cleavage domain. In some embodiments, the nuclease is a meganuclease. Examples of meganucleases include but are not limited to I-SceI, I-CreI, I-DmoI, and combinations thereof (e.g., E-DreI, DmoCre).

The term “CRISPR” refers to “clustered regularly interspaced short palindromic repeats”, which are DNA loci containing short repetitions of base sequences. CRISPR loci form a portion of a prokaryotic adaptive immune system that confers resistance to foreign genetic material. Each CRISPR loci is flanked by short segments of “spacer DNA”, which are derived from viral genomic material. In the Type II CRISPR system, spacer DNA hybridizes to transactivating RNA (tracrRNA) and is processed into CRISPR-RNA (crRNA) and subsequently associates with CRISPR-associated nucleases (Cas nucleases) to form complexes that recognize and degrade foreign DNA. In certain embodiments, the nuclease is a CRISPR-associated nuclease (Cas nuclease). Examples of CRISPR nucleases include, but are not limited to Cas9, dCas9, Cas6, Cpf1, and variants thereof. In some embodiments, the nuclease is Cas9. In some embodiments, the Cas9 is derived from the bacteria Streptococcus pyogenes (e.g., SpCas9) or Staphylococcus aureus (e.g., SaCas9). In some embodiments, a Cas protein or variant thereof does not exceed the packaging capacity of a viral vector, such as a lentiviral vector or an adeno-associated virus (AAV) vector, for example as described by Ran et al. (2015) Nature. 520(7546); 186-91. For example, in some embodiments, a nucleic acid encoding a Cas protein is less than about 4.6 kb in length.

For the purpose of genome editing, the CRISPR system can be modified to combine the tracrRNA and crRNA in to a single guide RNA (sgRNA) or just (gRNA). As used herein, the terms “guide RNA”, “gRNA”, and “sgRNA” refer to a polynucleotide sequence that is complementary to a target sequence in a cell and associates with a Cas nuclease, thereby directing the Cas nuclease to the target sequence. In some embodiments, a gRNA (e.g., sgRNA) ranges between 1 and 30 nucleotides in length. In some embodiments, a gRNA (e.g., sgRNA) ranges between 5 and 25 nucleotides in length. In some embodiments, a gRNA (e.g., sgRNA) ranges between 10 and 22 nucleotides in length. In some embodiments, a gRNA (e.g., sgRNA) ranges between 14 and 24 nucleotides in length. In some embodiments, a Cas protein and a guide RNA (e.g., sgRNA) are expressed from the same vector. In some embodiments, a Cas protein and a guide RNA (e.g., sgRNA) are expressed from separate vectors (e.g., two or more vectors).

Typically, a guide RNA (e.g., a gRNA or sgRNA) hybridizes (e.g., binds specifically to, for example by Watson-Crick base pairing) to a target sequence and thus directs the CRISPR/Cas protein or simple protein to the target sequence. In some embodiments, a guide RNA hybridizes to (e.g., targets) a nucleic acid sequence, e.g., within a C9ORF72 locus. In some embodiments, a guide RNA hybridizes to a target sequence on the sense strand (e.g., 5′-3′ strand) of a gene. In some embodiments, a guide RNA hybridizes to a target sequence on the antisense strand (e.g. 3′-5′ strand) of a gene.

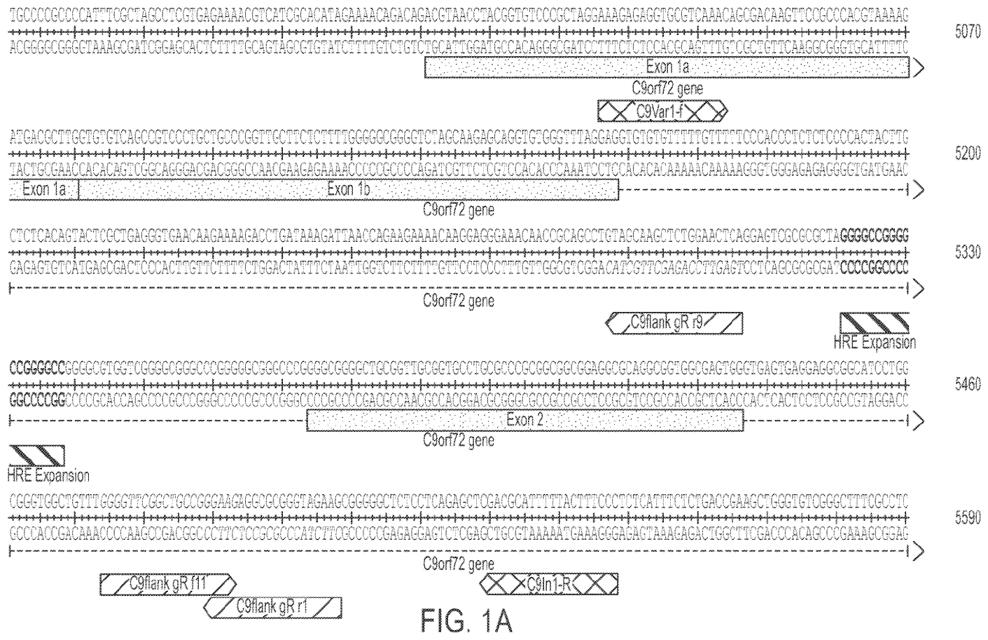

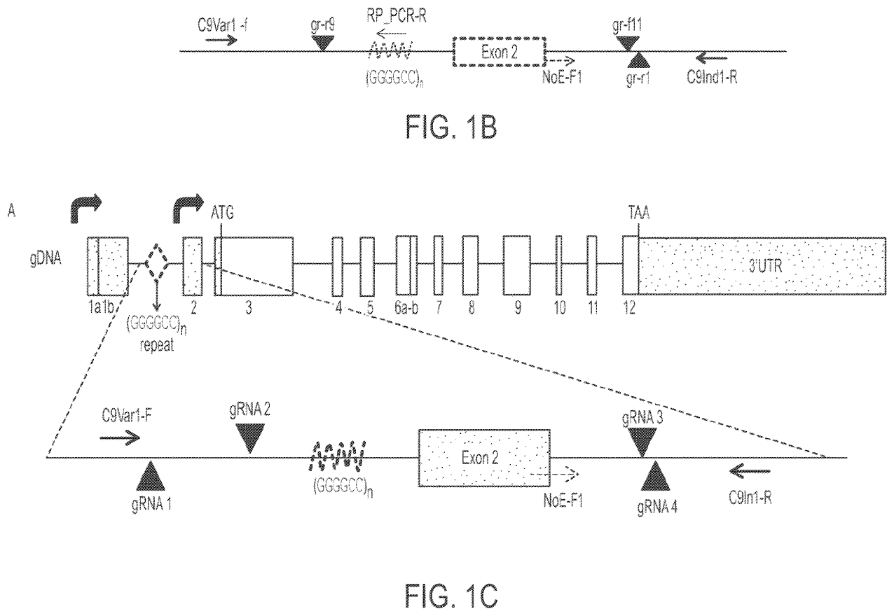

In some aspects, the disclosure relates to guide RNAs (gRNAs) that specifically hybridize to a target nucleic acid sequence flanking opposite sides of a G₄C₂ repeat of a C9ORF72 gene. As used herein “flanking opposite sides of a G₄C₂ repeat” refers to a first portion of a target nucleic acid sequence that is upstream (e.g., 5′) with respect to a G₄C₂ repeat and a second portion of a target nucleic acid sequence that is downstream (e.g., 3′) with respect to a G₄C₂ repeat (and also the first portion). For example, gRNA-R9 and gRNA-R1 represent a pair of gRNAs that specifically hybridize to a target nucleic acid sequence flanking opposite sides of a G₄C₂ repeat, as shown in FIG. 1A.

In some embodiments, a sequence that flanks a G₄C₂ repeat is positioned between 1 nucleotide and 1000 nucleotides (e.g., any integer between 1 and 1000) upstream (e.g., 5′) with respect to a G₄C₂ repeat (e.g., the first GGGGCC unit of the repeat). In some embodiments, a sequence that flanks a G₄C₂ repeat is positioned between 10 nucleotides and 800 nucleotides upstream (e.g., 5′) with respect to a G₄C₂ repeat. In some embodiments, a sequence that flanks a G₄C₂ repeat is positioned between 200 nucleotides and 700 nucleotides upstream (e.g., 5′) with respect to a G₄C₂ repeat. In some embodiments, a sequence that flanks a G₄C₂ repeat is positioned between more than 1000 nucleotides (e.g., 1500, 2000, 2500, 5000, or more) upstream (e.g., 5′) with respect to a G₄C₂ repeat.

In some embodiments, a sequence that flanks a G₄C₂ repeat is positioned between 1 nucleotide and 1000 nucleotides (e.g., any integer between 1 and 1000) downstream (e.g., 3′) with respect to a G₄C₂ repeat (e.g., the last GGGGCC unit of the repeat). In some embodiments, a sequence that flanks a G₄C₂ repeat is positioned between 10 nucleotides and 800 nucleotides downstream (e.g., 3′) with respect to a G₄C₂ repeat. In some embodiments, a sequence that flanks a G₄C₂ repeat is positioned between 200 nucleotides and 700 nucleotides downstream (e.g., 3′) with respect to a G₄C₂ repeat. In some embodiments, a sequence that flanks a G₄C₂ repeat is positioned between more than 1000 nucleotides (e.g., 1500, 2000, 2500, 5000, or more) downstream (e.g., 3′) with respect to a G₄C₂ repeat.

### Methods of Treatment

In some aspects, the disclosure provides methods for treating a subject having ALS or at risk of having ALS. A subject can be a human, non-human primate, rat, mouse, cat, dog, or other mammal.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms, for example, symptoms associated with ALS. Thus, the terms denote that a beneficial result has been conferred on a subject having ALS, or with the potential to develop such a disorder. Furthermore, treatment may include the application or administration of an agent (e.g., therapeutic agent or a therapeutic composition) to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease.

Therapeutic agents or therapeutic compositions may include a compound, vector, etc. in a pharmaceutically acceptable form that prevents and/or reduces the symptoms of a particular disease (e.g., ALS). For example a therapeutic composition may be a pharmaceutical composition that prevents and/or reduces the symptoms of ALS. In some embodiments, the disclosure provides a composition (e.g., a therapeutic composition) comprising one or more components of, or encoding, a gene editing complex as described by the disclosure, e.g., a vector as described by the disclosure. In some embodiments, the composition further comprises a pharmaceutically acceptable excipient. It is contemplated that the therapeutic composition of the present invention will be provided in any suitable form. The form of the therapeutic composition will depend on a number of factors, including the mode of administration as described herein. The therapeutic composition may contain diluents, adjuvants and excipients, among other ingredients as described herein.

### Pharmaceutical Compositions

In some aspects, the disclosure relates to pharmaceutical compositions comprising a gene editing complex. In some embodiments, the composition comprises gene editing complex and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known art. Except insofar as any conventional media or agent is incompatible with the active compound, use thereof in the compositions is contemplated. Supplementary active can also be incorporated into the compositions. Pharmaceutical compositions can be prepared as described herein. The active ingredients may be admixed or compounded with any conventional, pharmaceutically acceptable carrier or excipient. The compositions may be sterile.

Typically, pharmaceutical compositions are formulated for delivering an effective amount of an agent (e.g., gene editing complex). In general, an “effective amount” of an active agent refers to an amount sufficient to elicit the desired biological response. An effective amount of an agent may vary depending on such factors as the desired biological endpoint, the pharmacokinetics of the compound, the disease being treated (e.g., ALS), the mode of administration, and the patient.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. It will be understood by those skilled in the art that any mode of administration, vehicle or carrier conventionally employed and which is inert with respect in the active agent may be utilized for preparing and administering the pharmaceutical compositions of the present disclosure.

An effective amount, also referred to as a therapeutically effective amount, of a compound (for example, a gene editing complex or vector as described by the disclosure) is an amount sufficient to ameliorate at least one adverse effect associated with a condition (e.g., ALS). In the case of viral vectors, an amount of active agent can be included in each dosage form to provide between about 10¹⁰, 10¹¹, 10¹², 10¹³, 10¹⁴, or 10¹⁵ genome copies per subject. One of ordinary skill in the art would be able to determine empirically an appropriate therapeutically effective amount.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above.

The compositions may conveniently be presented in unit dosage forms. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. In some embodiments, liquid dose units are vials or ampoules. In some embodiments, solid dose units are tablets, capsules and suppositories.

### Modes of Administration

In some embodiments, a therapeutically effective amount of a gene editing complex or vector as described by the disclosure is delivered to a target tissue or a target cell. The pharmaceutical compositions containing gene editing complex or vector, and/or other compounds can be administered by any suitable route for administering medications. A variety of administration routes are available, including parenterally, intravenously, intrathecally, intracranially, intradermally, intramuscularly or subcutaneously, or transdermally. The methods of this disclosure, generally speaking, may be practiced using any mode of administration that is medically acceptable, meaning any mode that produces therapeutic effect without causing clinically unacceptable adverse effects. Various modes of administration are discussed herein. For use in therapy, an effective amount of the gene editing complex or vector, and/or other therapeutic agent can be administered to a subject by any mode that delivers the agent to the desired tissue, e.g., systemic, intramuscular, etc. In some embodiments, the gene editing complex or vector as described by the disclosure is administered to a subject via intramuscular (IM) injection or intravenously.

In some embodiments, a gene editing complex (e.g., a nucleic acid encoding one or more components of a gene editing complex) can be delivered to the cells via an expression vector engineered to express the gene editing complex. An expression vector is one into which a desired sequence may be inserted, e.g., by restriction and ligation, such that it is operably joined to regulatory sequences and may be expressed as an RNA transcript. An expression vector typically contains an insert that is a coding sequence for a protein (e.g., gene editing protein, such as a CRISPR/Cas protein) or for a polynucleotide, such as guide RNA (gRNA, sgRNA, etc.). Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays or fluorescent proteins, etc.

As used herein, a coding sequence (e.g., protein coding sequence, miRNA sequence, shRNA sequence) and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. It will be appreciated that a coding sequence may encode an functional RNA.

The precise nature of the regulatory sequences needed for gene expression may vary between species or cell types, but shall in general include, as necessary, 5′ non-transcribed and 5′ non-translated sequences involved with the initiation of transcription and translation, respectively, such as a TATA box, capping sequence, CAAT sequence, and the like. Such 5′ non-transcribed regulatory sequences will include a promoter region that includes a promoter sequence for transcriptional control of the operably joined gene. However, in some embodiments, a vector does not include a promoter sequence. Regulatory sequences may also include enhancer sequences, upstream activator sequences, internal ribosomal entry sites (IRES), and/or self-processing peptide sequences (e.g., 2A peptide), as desired. The vectors of the disclosure may optionally include 5′ leader or signal sequences.

In some embodiments, a virus vector for delivering a nucleic acid molecule is selected from the group consisting of adenoviruses, adeno-associated viruses, lentiviral vectors, etc. In some embodiments, the viral vector is a recombinant adeno-associated virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. The adeno-associated virus can also function in an extrachromosomal fashion.

In some embodiments, a recombinant AAV vector (rAAV) comprises, at a minimum, a transgene coding sequence (e.g., a nucleic acid sequence encoding a gene editing protein, such as a Cas protein, or a gRNA) and its associated regulatory sequence flanked by two AAV inverted terminal repeat (ITR) sequences. Examples of regulatory sequences include promoters (e.g., constitutive promoters, inducible promoters, tissue-specific promoters), enhancer sequences, etc. In some embodiments, the ITR sequences are AAV1, AAV2, AAV5, AAV6, AAV7, AAV8, or AAV9 ITR sequences, or variants thereof.

In some embodiments, an rAAV vector comprising a nucleic acid encoding all or part of a gene editing complex (e.g., a nucleic acid sequence encoding a gene editing protein, a gRNA, or both) is packaged into a recombinant AAV (rAAV). Typically, an AAV vector is packaged into viral particles comprising one or more AAV capsid proteins. In some embodiments, the AAV capsid is an important element in determining these tissue-specific targeting capabilities. Thus, an rAAV having a capsid appropriate for the tissue being targeted can be selected. In some embodiments, the capsid protein has a serotype selected from AAV2, AAV3, AAV5, AAV6, AAV6.2. AAV7, AAV8, AAV9, AAVrh.8, AAVrh.10, AAVrh.39, and AAVrh.43 or suitable variants of any one of them. In some embodiment, the rAAV comprises a capsid protein that targets neuronal cells.

In some embodiments, other useful viral vectors are based on non-cytopathic eukaryotic viruses in which non-essential genes have been replaced with the gene of interest. Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler. M., “Gene Transfer and Expression, A Laboratory Manual,” W. H. Freeman Co., New York (1990) and Murry. E. J. Ed. “Methods in Molecular Biology,” vol. 7, Humana Press. Inc., Clifton, New Jersey (1991). In some embodiments, gene editing complex (e.g., a nucleic acid sequence encoding a gene editing protein, a gRNA, or both) is delivered to a cell (e.g. a cell of a subject) by a lentiviral vector.

Various techniques may be employed for introducing nucleic acid molecules of the disclosure into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER™ Nanoparticle Transfection System by Sigma-Aldrich, FectoFly™ transfection reagents for insect cells by Polyplus Transfection, Polyethylenimine “Max” by Polysciences, Inc., Unique, Non-Viral Transfection Tool by Cosmo Bio Co., Ltd., Lipofectamine™ LTX Transfection Reagent by Invitrogen, SatisFection™ Transfection Reagent by Stratagene, Lipofectamine™ Transfection Reagent by Invitrogen, FuGENE® HD Transfection Reagent by Roche Applied Science, GMP compliant in vivo-jetPET™ transfection reagent by Polyplus Transfection, and Insect GeneJuice® Transfection Reagent by Novagen.

### EXAMPLES

### Example 1: Excision of G₄C₂ Expansion

### Strategy Design and Testing in HEK Cells.

This example describes removal of the G₄C₂ expansion repeat in C9Orf72 using a CRISPR/Cas9 system. Several guide RNAs targeting the flanking regions of the G₄C₂ expansion were designed. The G₄C₂ expansion and guide RNAs are shown in FIG. 1A. Guides determined to be successful in achieving significant editing, as described herein, are shown. In order to test gene editing events, two primers, C9Var1-F and C9In1-R, that span the repeat expansion and the guides were designed (FIGS. 1A-1C). These primers can amplify through few repeats, but will generally not amplify through the 45-60 repeats present in the BAC436 mouse model. In order to detect no editing in the BAC436 model, a NoE-F1 primer that can in conjugation with C9In1-R-amplify ˜120 bp band in unedited DNA, was designed (FIGS. 1B-1C). Another primer that recognizes the GGGGCC sequence within the repeat was designed (FIGS. 1B-1C). This primer was used for the repeat primed PCR (RP-PCR) described herein.

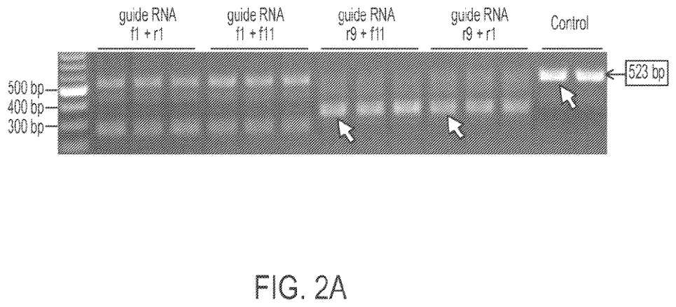

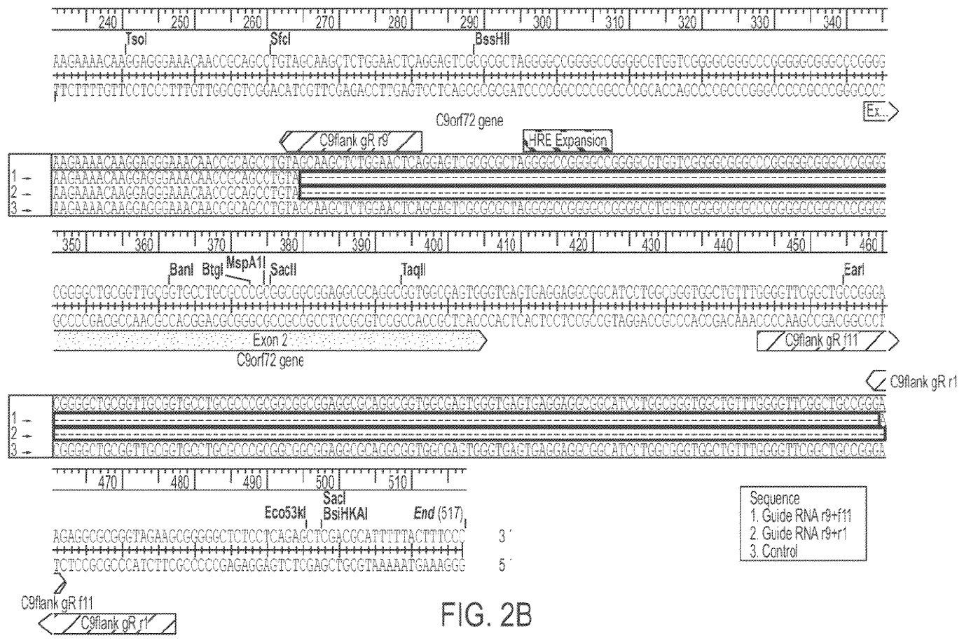

Four different guide RNA constructs, two on the 5′ end of the repeat expansion (f1, also referred to as “gRNA1” & r9, also referred to as “gRNA 2”) and two on the 3′ end (r1, also referred to as “gRNA 4”& f11, also referred to as “gRNA 3”) (Table 1), were generated. Then, plasmids expressing two of each guides as follows were generated: gRNA f1-r1, gRNA f1-f11, gRNA r9-f11, gRNA r9-r1. Each of these plasmids was co-transfected into HEK293T cells with another plasmid expressing S. pyogenes Cas9. DNA was extracted from these HEK 293T cells and a PCR was performed using C9Var1-F and C9In1-R. The products were run on an agarose gel (FIG. 2A). In case no editing occurs, these primers will amplify a 523 bp band. In case editing occurs, gRNA f1-r1 and f1-f11 will produce a ˜250 bp band while r9-f11 and r9-r1 will produce a ˜320 bp band.

TABLE 1
Guide RNAs generated for
“Excision of G₄C₂ expansion.”

| guide RNA name | guide RNA sequence | SEQ ID NO: |
|---|---|---|
| gRNA-f11 | GGGGUUCGGCUGCCGGGAAG | 1 |
| gRNA-r1 | GGAAGAGGCGCGGGUAGAAG | 2 |
| gRNA-r9 | GUAGCAAGCUCUGGAACUCA | 3 |
| gRNA-f1 | UGCUCUCACAGUACUCGCUG | 4 |

As seen on the gel (FIG. 2A) these four different combination are capable of editing C9 gene in HEK cells, since bands of the anticipated edited size in each of these guide RNA combinations are observed. gRNA f1-r1 and gRNAf1-f11 both have a faint band between 200 and 300 bp, while gRNA r9-f11 and gRNA r9-r1 have a strong band around 320 bp. Both of these bands are absent in the untreated control. However, the combination of r9-f11 and r9-r1 seems to be much more efficient at gene editing, since the edited band is much more intense then f1-r1 and f1-f11 alone. Additionally, the unedited band at 523 bp is almost completely gone from r9-f11 and r9-r1. Bands labeled with arrow heads in FIG. 2A were then extracted and sequenced to ensure that gene editing occurred at the expected locations (FIG. 2B). Based on this data an AAV9 virus containing gRNA r9-r1 and r9-f11 was generated to use for the in vivo studies.

### C9ORf22 Gene Editing in Mice Primary Neurons

A mouse model (Bac436) expressing human C9orf72 with 45-65 expanded GGGGCC repeats has been developed. This model contains 6-8 copies of the C9orf72 gene in heterozygous (het) animals and 12-16 copies in homozygous (homo) animals. Additionally, a mouse expressing Cas9 gene, in addition to C9orf72 with the expansion, is observed in this model. In order to determine whether guides will successfully excise the GGGGCC repeat in mice primary neurons, appropriate crosses of the BAC436 mice expressing C9orf72 and Cas9 were set up to produce only heterozygous progeny. Primary neurons were isolated at embryonic day 14 (E14), and cultured appropriately. After 4 days in culture, neurons were either treated with PBS alone, or infected with AAV9 CB-GFP, AAV9 SOD1 guide RNA (control guide), AAV9-CB-GFP. C9gR flank r9-r1, or AAV9-CB-GFP-C9gR flank r9-f11. At 72 hours, 25,000 MOI was recorded and the cells were harvested. The DNA was isolated using QIAGEN™ blood and tissue DNA extraction kit.

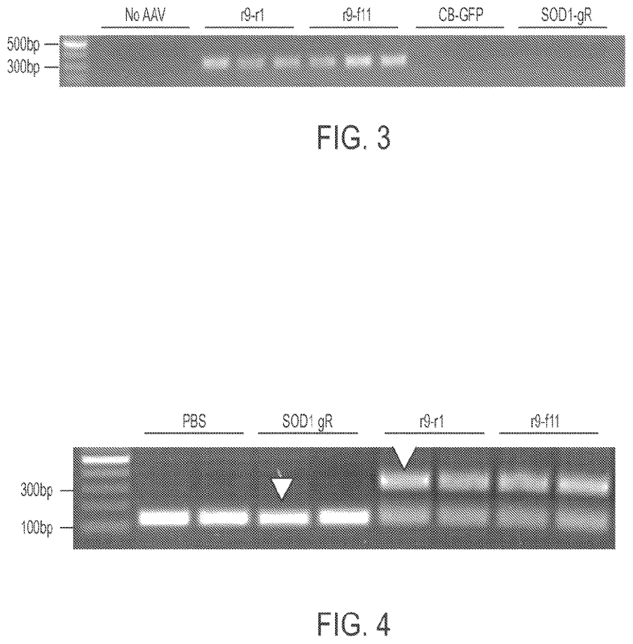

In order to determine whether editing has occurred in these isolated neuronal cells, a PCR reaction was performed using C9Var1-f and C9In1-R (FIGS. 1A and 1B). Without gene editing, these primers fail to amplify through the repeat and no band appears on the gel. When gene editing occurs, the repeat is excised out and primers amplify a single band at 321 bp. In both sets of guides a strong band appearing at the right size is observed, while this band is absent in both non-AAV treated neurons and those transfected with CB-GFP, or SOD1-gR (FIG. 3).

### Testing Guide RNA Constructs in Mice Livers

In order to determine whether gene editing is also successful in vivo, four groups of Cas9/+, C9/+ mice were tail vein injected with PBS alone, AAV9 SOD1 guide RNA, AAV9-CB. GFP-C9gR flank r9-r1, or AAV9-CB-GFP-C9gR flank r9-f11. Two weeks after injection, mice were sacrificed and tissues were harvested. Since tail vein injection is very efficient at transfecting liver cells, DNA isolated from liver was analyzed. A third primer (NoE-F1) that can amplify unedited DNA, in conjugation with C9In1-R, was designed (FIG. 1B). To reduce competition between C9Var1-f and NoE-FL two different PCR reactions were run separately with C9Var1-f and C9In1-R or NoE-F1 and C9In1-R. Products from these two PCRs were mixed and run on the same gel (FIG. 4). A 321 bp band appears in samples from mice injected with AAV9-CB-GFP-C9gR flank r9-r1 and AAV9-CB-GFP-C9gR flank r9-f11, but not from mice injected with AAV9 SOD1 guide RNA or PBS alone (FIG. 4). Moreover. the 100 bp amplified by NoE-F1 and C9In1-R from unedited DNA was much less intense in r9-r1 and r9-f11 mice in comparison to control mice. The labeled bands were isolated and sequenced to confirm that the correct size gene editing products were made.

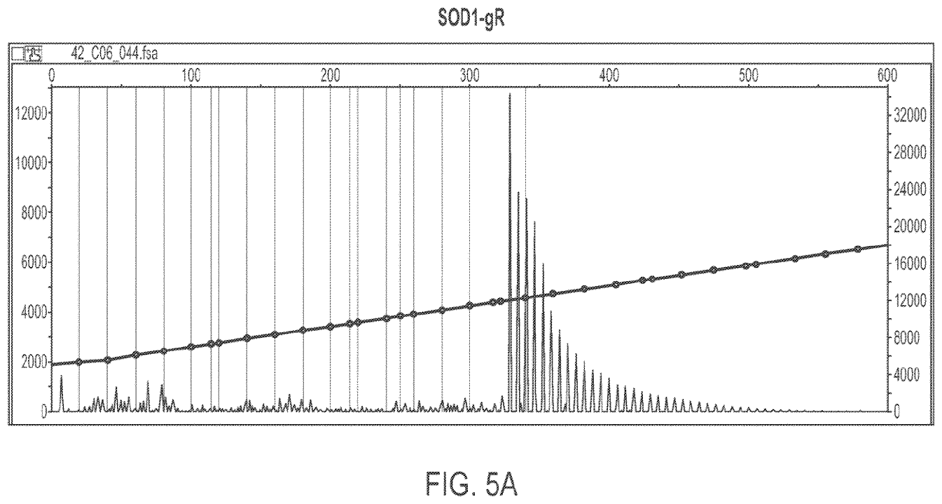

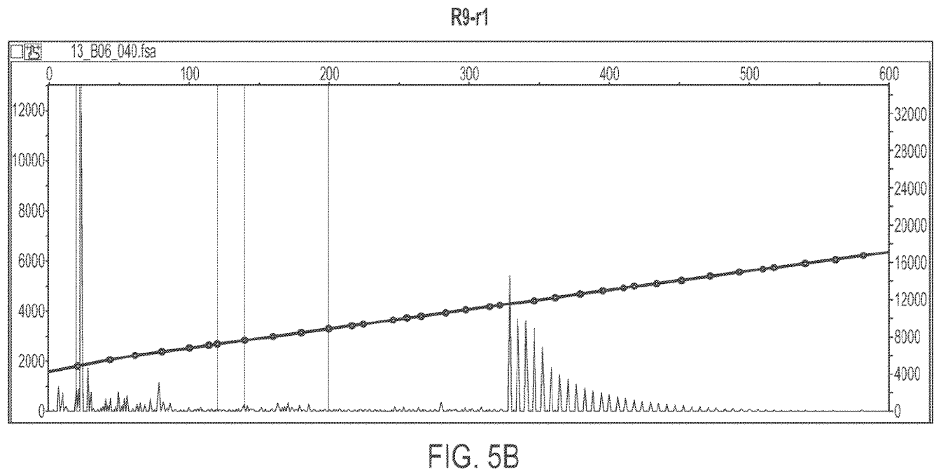

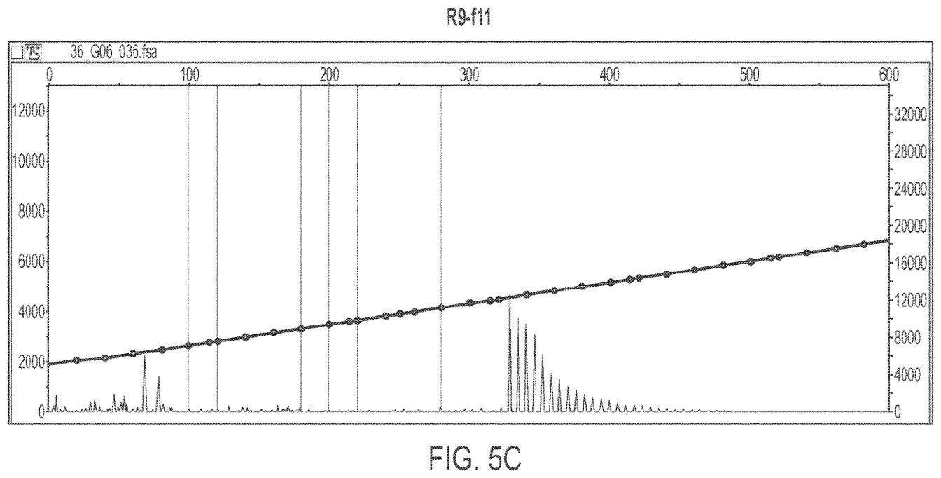

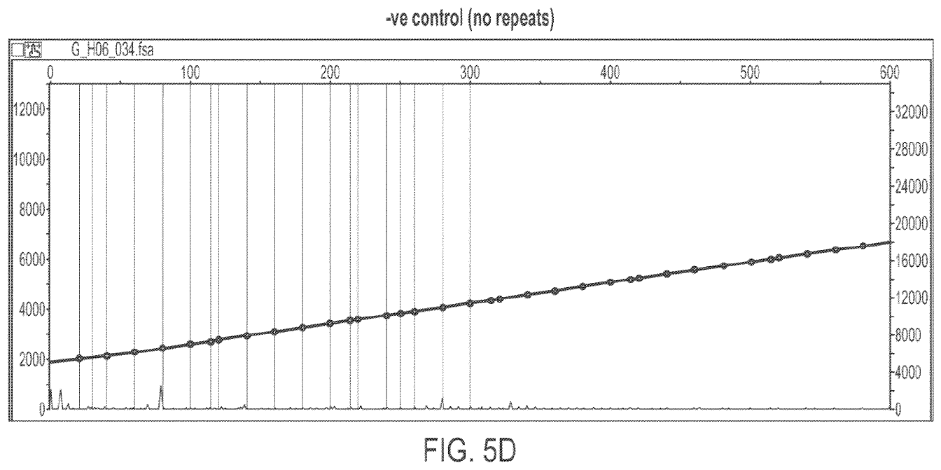

To further elucidate editing, a Repeat Primed PCR was performed using a FAM-tagged C9Var1-f and c9ccccggLCM13F_MRX-R1b. The latter is a reverse primer that recognizes and binds the GGGGCC repeat. This form of PCR reaction produces different sized fragments based on where in the repeat the reverse primer binds and starts the amplification. These fragments were then analyzed on a fragment analyzer to produce an electropherogram where each peak reflects a different sized fragment and its intensity reflects fragment abundance. As the primer binds deeper into the repeat, it becomes more difficult to amplify and thus the intensity of peaks on the electropherogram decreases with larger fragments. These fragments can only be amplified in unedited DNA, and the shortest most intense fragment is around 330 bp in size. The electropherograms of the Repeat primed PCR products for AAV9 SOD1 guide RNA, AAV9. CB-GFP-C9gR flank r9-r1, AAV9-CB-GFP-C9gR flank r9-f11, and uninjected wild type C57BL mice that don't express human C9 are shown in FIGS. 5A-5D, respectively. The results confirm Cas9-mediated C9orf72 G4C2 editing in vivo.

### Example 2: Induction of Non-Sense Mediated Decay of C9orf72 Transcripts

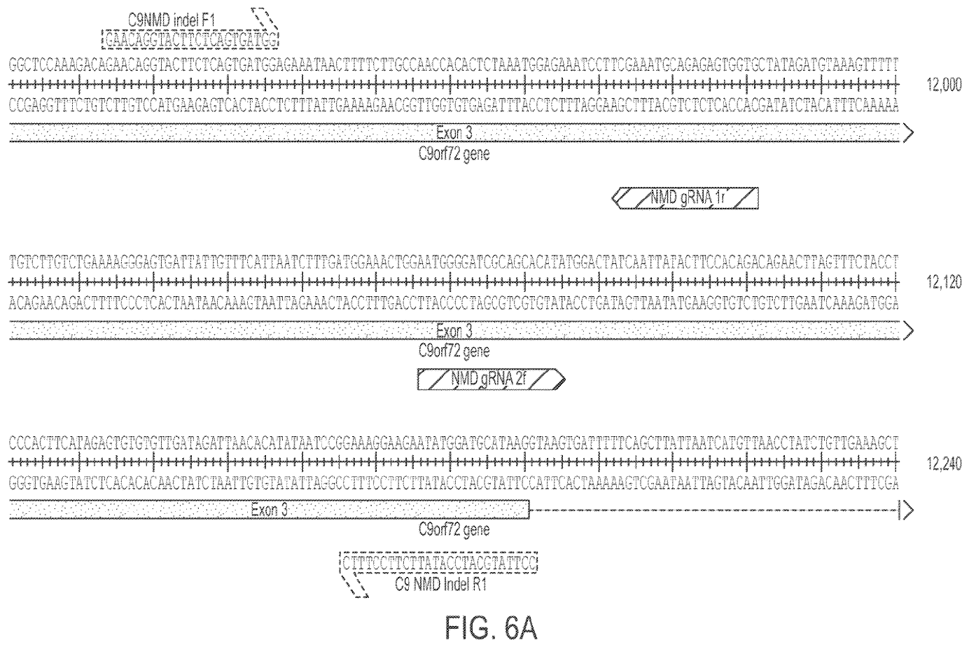

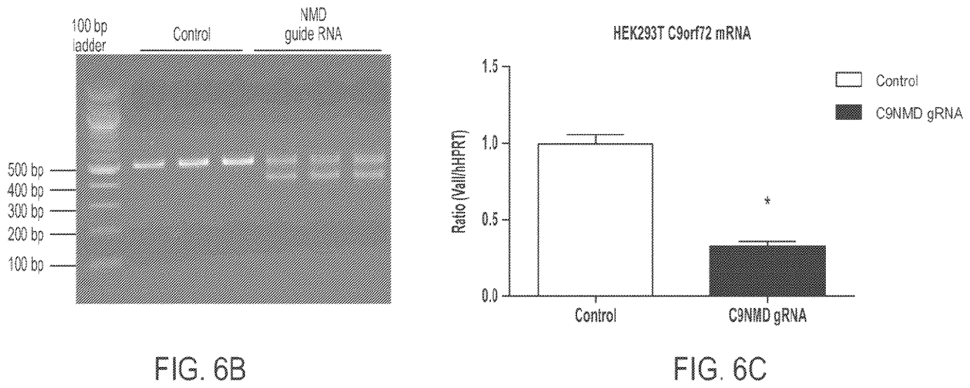

In this example, guide RNAs were designed to target exon 3 after the ATG initiation codon of C9orf72 (Table 2). The strategy was to introduce small indels that will lead to early termination codon, thus inducing non-sense mediated decay of C9orf72 transcripts to reduce RNA foci and dipeptide formation. FIG. 6A shows the human C9orf72 gene sequence of exon 3 with the locations of the non-sense mediated decay (NMD) guide RNA 1r and 2f and the location and sequence of PCR indel analysis primers C9NMD Indel F1 and R1 marked. FIG. 6B shows the results of agarose gel electrophoresis of the PCR products amplified by the C9NMD-Indel F1 and R1 PCR primers. In this example, HEK293T cells were transfected with LV-SpCas9 (Control) or LV-NMDgR-SpCas9 plasmid (2 μg) in triplicate. FIG. 6C shows the results of digital droplet PCT (ddPCR) analysis of the C9orf72 RNA levels from FIG. 6B.

TABLE 2
Guide RNAs generated for
“Non-sense mediated decay.”

| | | SEQ |
|---|---|---|
| | | ID |
| guide RNA | guide RNA sequence | NO: |
| NMD gRNA 1r | UCGAAAUGCAGAGAGUGGUG | 5 |
| NMD gRNA 2f | AAUGGGGAUCGCAGCACAUA | 6 |

### Example 3: Direct Visualization of C9ORf72 Gene Editing in Primary Neurons

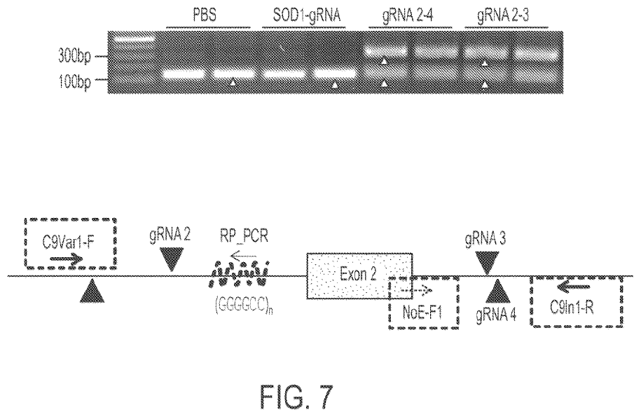

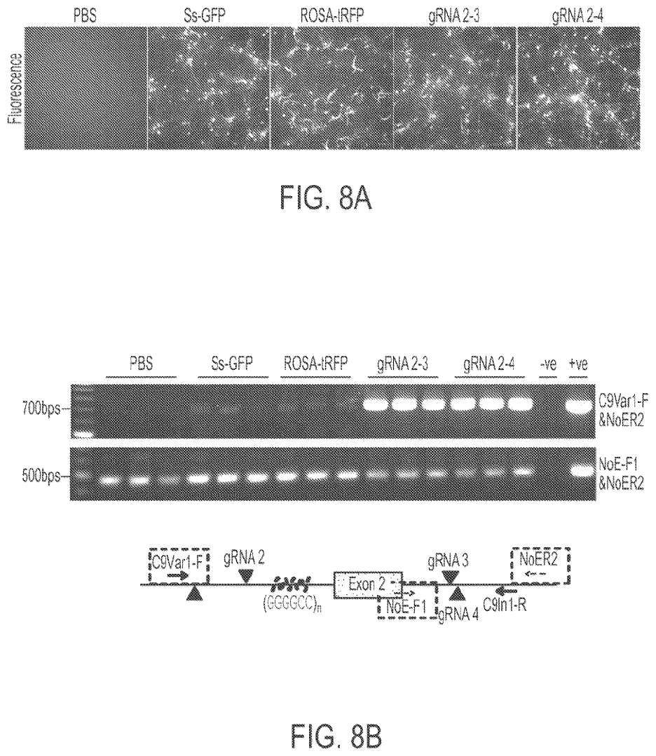

A mouse model (BAC111) expressing human C9orf72 with 45-65 expanded GGGGCC repeats has been developed. This model contains 6-8 copies of the C9orf72 gene in heterozygous (het) animals and 12-16 copies in homozygous (homo) animals. Additionally, this mouse model expresses Cas9, in addition to C9orf72 with the expansion. In order to determine whether guides successfully excise the GGGGCC repeat in mice primary neurons, appropriate crosses of the BAC111 mice expressing C9orf72 and Cas9 were set up to produce only heterozygous progeny. Primary neurons were isolated at embryonic day 14 (E14), and cultured appropriately. After 4 days in culture, neurons were either treated with PBS alone, or infected with AAV9 single-stranded-GFP (ss-GFP), AAV9-ROSA-tRFP guide RNA (control guide), AAV9-GFP-C9gR flank gRNA 2 & 3, or AAV9-GFP-C9gR flank gRNA 2 & 4. At 72 hours, 25,000 MOI was recorded and the cells were harvested. The DNA was isolated using QIAGEN™ blood and tissue DNA extraction kit. PCR results are shown in FIG. 7. The cultured primary neurons were imaged for GFP or REP fluorescence to visualize the incorporation of AAV9-gRNA constructs to into primary neurons (FIG. 8A).

In order to determine whether editing occurred in these isolated neuronal cells, a PCR reaction was performed using C9Var1-F and NoER2 primers (FIG. 8B). Without gene editing, these primers fail to amplify through the repeat and no band appears on the gel. When gene editing occurs, the repeat is excised out and primers amplify a single band at about 720 base pairs. In both sets of guides a strong band appearing at the right size is observed, while this band is absent in both non-AAV treated neurons (PBS) and those transfected with ss-GFP, or ROSA-tRFP (FIG. 8B). In order to estimate the level of unedited DNA, a PCR reaction was performed using NoE-F1 and NoER2 (FIG. 8B). A band of about 500 base pairs appears on a gel when gene editing has not occurred. Control gene editing conditions (PBS, ss-GFP, or ROSA-tRFP) produced an intense band at about 500 base pairs, while both sets of gRNA 2 & 3 and gRNA 2 & 4 guides have less unedited samples.

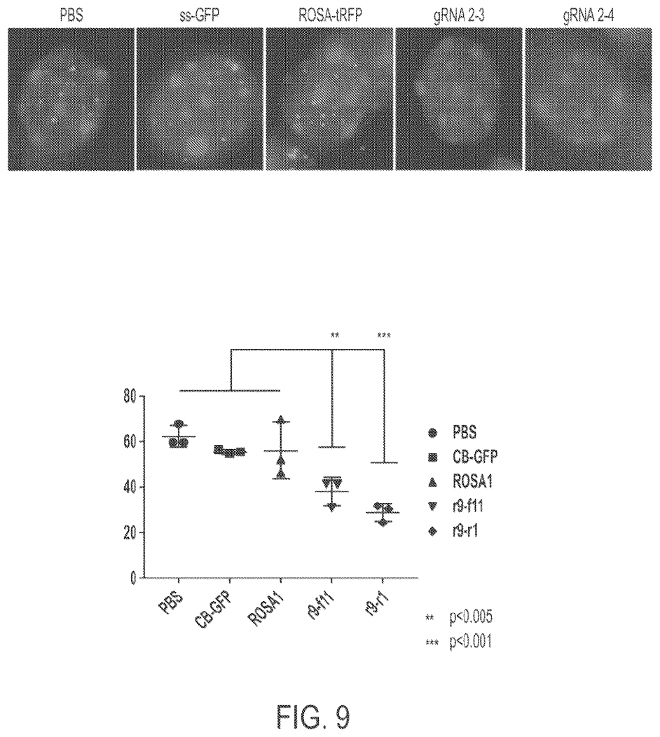

To directly visualize gene editing, cultured primary neurons from BAC111 mice expressing human C9orf72 and Cas9 were isolated and treated with PBS, AAV9-ss-GFP, AAV9-ROSA-tRFP, AAV9-gRNA 2 & 3, AAV9-gRNA 2 & 4 as above. Fluorescence in situ hybridization (FISH) was used to visualize unedited C9orf72 RNA (punctate staining, e.g., foci) and nuclei were stained with DAPI (FIG. 9). Almost 55-60% of unedited cells have more than ten foci, while edited cells exhibit significantly less in only 35-40% of cells (FIG. 9).

### Example 4: Exogenous Cas9 Promotes C9ORf72 Gene Editing in Primary Neurons

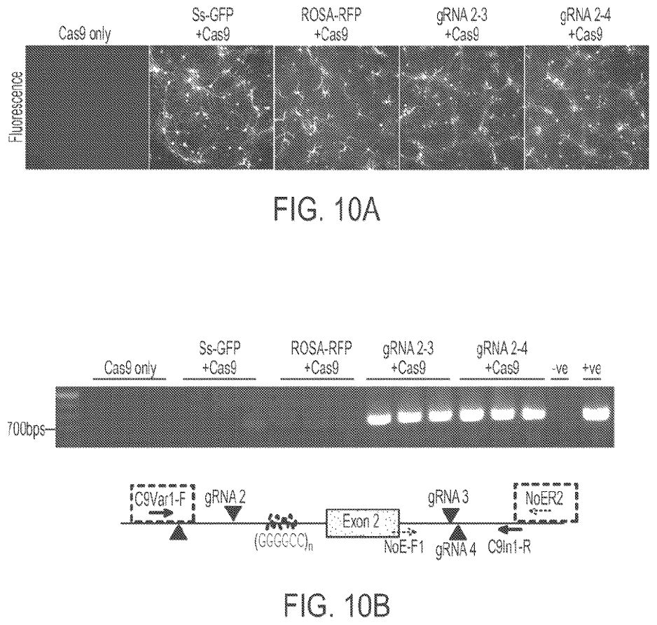

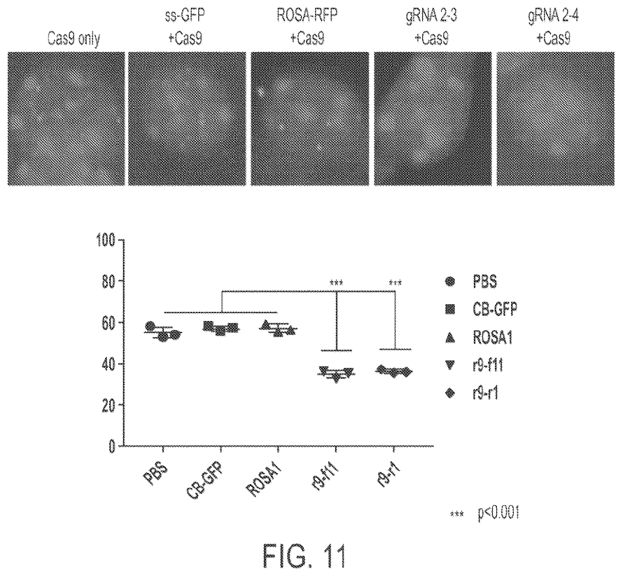

To directly test whether C9orf72 excision of GGGGCC repeats requires endogenous Cas9 expression, BAC111 mouse models expressing C9orf72 and nor Cas9 were produced. Primary neurons were isolated at embryonic day 14 (E14), and cultured appropriately. After 4 days in culture, neurons were supplemented with Cas9 and either treated with Cas9 alone, or infected with AAV9-ss-GFP+Cas9, AAV9-ROSA-RFP+Cas9 (control guide), AAV9-GFP-C9gR flank gRNA 2 & 3, or AAV9-GFP-C9gR flank gRNA 2 & 4. At 72 hours, 25,000 MOI was recorded and the cells were harvested. The DNA was isolated using QIAGEN™ blood and tissue DNA extraction kit. The cultured primary neurons were imaged for GFP or RFP fluorescence to visualize the incorporation of AAV9-gRNA constructs to into primary neurons (FIG. 10A).

PCR amplification of edited DNA from cultured neurons was performed. Briefly, edited DNA was amplified by PCR with C9Var1-F & NoER2 (FIG. 10B). Amplification hands occur only in edited cells (e.g., cells treated with AAV9-gRNA 2-3+Cas9, or AAV9-gRNA 2-4+Cas9), as shown in FIG. 10B.

FIG. 11 shows direct visualization and quantification of gRNAs bound to unedited DNA from primary cultured neurons isolated from BAC111 mice expressing C9 by FISH. Around 55-60% of cells have foci when unedited (Cas9 only, single stranded GFP, ROSA). Foci in edited cells were reduced to 35-40%. Treatment with both gRNA pairs resulted in a significantly different reduction.

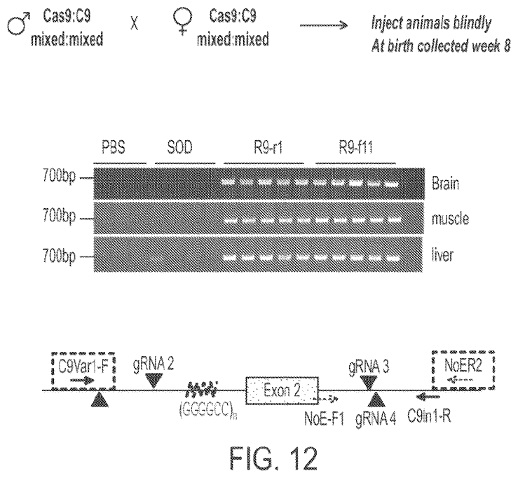

Tissue distribution of gene editing constructs (e.g., rAAVs) was examined. FIG. 12 shows gene editing in vivo in BAC111 mice expressing C9/Cas9 injected with PBS, SOD gRNA (control), R9-r1 (gRNA 2 & 4), or R9-f11 (gRNA 2 & 3). Brain, muscle, and liver tissue samples taken after 8 weeks each demonstrated gene editing with gRNA 2 & 3 and gRNA 2 & 4 guides, but not PBS and control SOD gRNA.

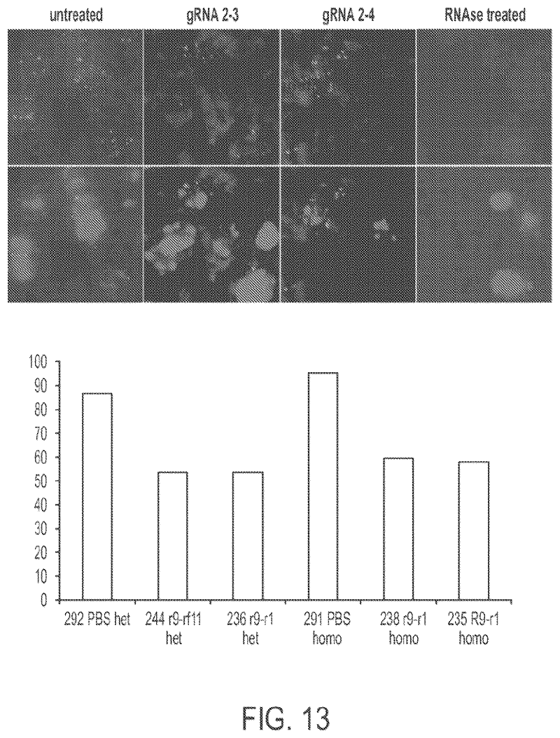

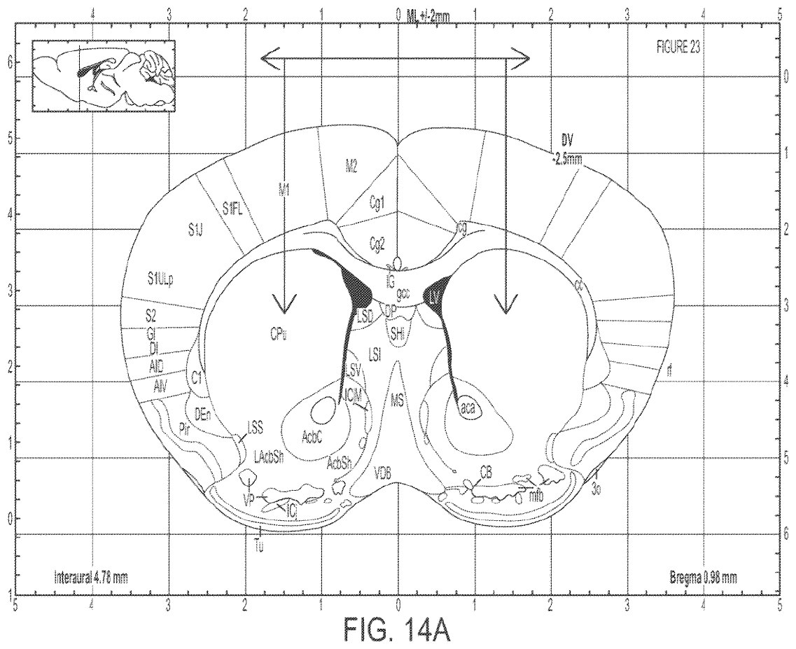

FIG. 13 shows FISH data (sense direction) on frontal sections of CAC111 mice that were facially injected at p1-2. The top panel shows a fluorescence micrograph indicating a reduction in number of foci in edited cells compared to untreated and control cells. The bottom panel shows data indicating the reduction is consistent for heterozygous and homozygous mice.

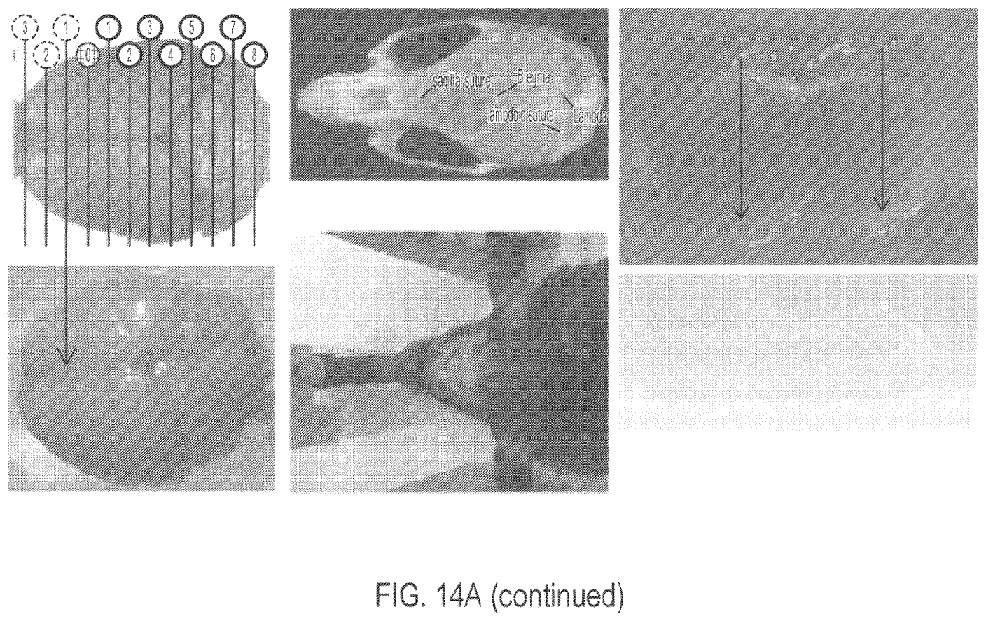

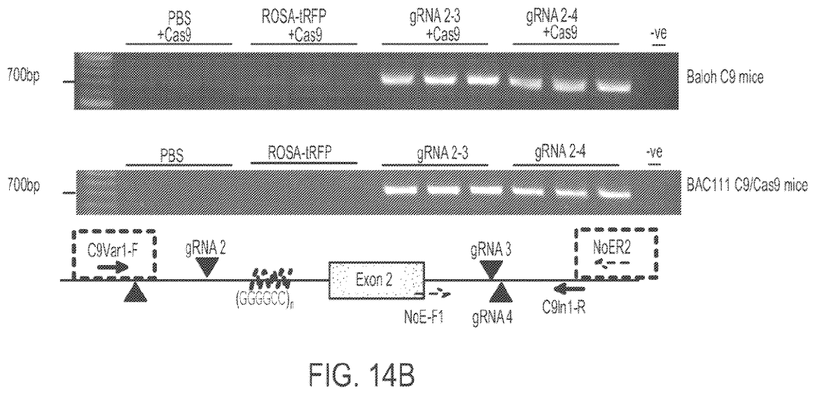

FIGS. 14A-14B show gene editing through stereotaxic striatal brain injections in Baloh and BAC111 mice. FIG. 14A shows the injection site and the brain slice used for tissue isolation. FIG. 14B shows that injection of PBS+Cas9, ROSA-tRFP+Cas9, gRNA 2 & 3+Cas9, gRNA 2 & 4+Cas9 promotes gene editing in Baloh C9 mice and BAC111 C9/Cas9 mice.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively. as set forth in the United States Patent Office Manual of Patent Examining Procedures, Section 2111.03.

Use of ordinal terms such as “first,”second,“third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1.-9. (canceled)

10. A recombinant adeno-associated virus (rAAV) comprising:
(i) the isolated nucleic acid comprising a transgene encoding a first guide RNA (gRNA) and a second gRNA that each specifically hybridize to a target nucleic acid sequence flanking opposite sides of a G4C2 repeat of a C9ORF72 gene, flanked by adeno-associated virus (AAV) inverted terminal repeats (ITRs); and
(ii) at least one AAV capsid protein.

11. The rAAV of claim 10, wherein the capsid protein is of a serotype selected from AAV1, AAV2, AAV3, AAV4, AAV5, AAV6, AAV7, AAV8, AAV9, or a variant of any of the foregoing.

12. The rAAV of claim 10, wherein the capsid protein is an AAV9 capsid protein.

13. A composition comprising the rAAV of claim 10, and a recombinant gene editing protein.

14. The composition of claim 13, wherein the recombinant gene editing protein is encoded by an rAAV vector.

15. The composition of claim 13, wherein the recombinant gene editing protein is a CRISPR/Cas protein, optionally a Cas9 protein.

16.-55. (canceled)

56. The rAAV claim 10, wherein the transgene encodes a first gRNA having the sequence set forth in SEQ ID NO: 2 and a second gRNA having the sequence set forth in SEQ ID NO: 3.
